(19) United States
(12) Patent Application Publication
(10) Pub. No.: US 2021/0267349 A1
(43) Pub. Date: Sep. 2, 2021

(54) MULTIFUNCTIONAL PENCIL FOR EYEBROW STYLING

(71) Applicant: PHI Academy DOO, Belgrade (RS)

(72) Inventor: Branko Babic, Belgrade (RS)

(21) Appl. No.: 17/260,542

(22) PCT Filed: Jul. 4, 2019
(86) PCT No.: PCT/IB2019/055712

§ 371 Date: Jan. 14, 2021

(30) Foreign Application Priority Data
Aug. 21, 2018 (RS) P-2018/0980

(51) Int. Cl. A45D 34/04

(52) U.S. Cl. CPC A45D 34/04

(57) ABSTRACT

The multifunctional pencil for eyebrow styling enables styling of eyebrows by means of a pencil that in itself contains two types of blades, an U-shaped blade for the drawing of hairs, and a blade in the form of a needle used to shade the space between the hairs. The multifunctional pencil (1) for eyebrow styling has a cylindrical lid (2) which is placed on the middle part (3) of the pencil (1) which has a blade (4) on its front and on its rear there is an opening for the needle insert (5) located on the front side of the rear part (6), which at the end has a rectangular hygienic applicator (7) that has three ribs.

### FIELD OF THE INVENTION

The subject invention relates to a multifunctional pencil for eyebrow styling. According to the International Patent Classification, the subject invention belongs to the tools for introducing a medium, in this case a dye, under the skin, which are designated with code A61M 37/00.

### TECHNICAL PROBLEM

The technical problem solved by the subject invention consists of how to design a pencil for the treatment of eyebrow styling, which would contain two types of blades in itself: such as the U-shaped blade, used for drawing the hairs, and a blade in the shape of a needle, which is used to shade the space between the hairs, so that it constitutes a unique tool that would be easy to handle and that would shorten the time of work, at which the blades would be housed in the pencil itself, which would prevent contamination as the complete pencil would be sterile.

### PRIOR ART

Even in the past, for better appearance, women began to pencil in their eyebrows and thus shape and modify them. This was originally done with eyebrow styling pencils and by tweezing and waxing. With the industry's development in terms of beauty products, the need for perfect eyebrows has also developed. So today, there are various methods and techniques for modifying and shaping eyebrows such as tattooing, dyeing, make-up, re-shaping by hair removal, and the like. However, one technique that has taken the primacy in the filling and shaping eyebrows is—microblading.

Microblading is a technique for filling and shaping eyebrows that gives eyebrows fullness and natural form without additional styling and pencil shading. This technique is able to complement existing eyebrows, correct irregularities, or draw a completely new shape. It does not require removal of existing hair, but it follows the line of the natural arch of the eyebrows and fills-in empty space with an aim of achieving a beautiful contour and fullness of the eyebrows. To fill-in an eyebrow, a mineral pigment in liquid, creamy or gel form is used and applied to the area of the eyebrow. During the treatment, the pigment is inserted shallow under the skin by means of the blade and in this manner the eyebrows are drawn. At which two techniques are combined: microblading and shading. This does not fill-in the whole eyebrow, but rather hairs are drawn together with natural hairs with a dye that is not different from the existing color of natural hair, which gives eyebrows a natural appearance.

The difference between the existing styling methods, such as tattooing, in relation to the microblading technique, by application of the subject-matter invention, is in the direction, density, length and thickness of the drawn hair. Namely, tattooing is exclusively based on the filling of the brow area, whereby after a while, dye spills and irregularities can occur, and in some cases the pigment takes on a shade of cyan. Conversely, microblading takes particular care of the position of the very hair that are plotted and which are very thin, which provides a more natural look.

The tool most commonly used to plot eyebrows with various permanent make-up methods is a small hand-held single-needle machine. In practice, there are several enhanced versions of this device, however, the point is that it uses a needle that, once dipped into the pigment, penetrates into the dermal layer of the skin, resulting in the spread of dye also into the area that should not be dyed, which results in spillage and irregular blurred lines.

In microblading, a specially designed tool is used, i.e. a fine, special blade that is placed in the holder. The blade itself is made of many fine, tiny pins that are used to perform extremely delicate and precise cuts into the skin so that it may absorb the pigment that will ultimately give the appearance of natural eyebrow hairs. A blade such as this guarantees the highest possible precision of pigment implementation so far, and provides a gentle and precise contours without causing unintended side effects that occur in tattooing; conversely, all lines are streaked precisely and mimic the appearance of natural hairs.

Until now, people used holders to alternatively use blades or pins, depending on which technique is being used, i.e. whether they wanted to perform microblading or shading. Each technique requires a certain blade, which consumes time during the exchange and thus prolongs the duration of the treatment. The subject invention shortens the time working time because the blades are situated in one package, meaning in one pencil, which is an accelerated work mode due to the simplified transition from one blade to the other.

### SUMMARY OF THE INVENTION

The above-defined technical problem is solved by a pencil that carries a microblading blade, a shadow blade, and a hygienic applicator. The pencil itself consists of a lid, a middle part which has one opening in the front and one in the back, and a rear part that has a shading needle in the front and a hygienic applicator in the back.

The cover is cylindrical in shape, made of transparent plastic so that the operator would have a visual overview of the blade itself. On the front of the cover there is a round cone opening for penetration of the sterilized gas to the blade, while the back part of the lid is round-shaped with a ring around the entire inner rim, the function of which is to position the lid for the middle part of the pencil.

The middle part of the pencil is barrel-shaped with two cylindrical surfaces that give symmetry to the piece and serve as an aesthetic and functional connection of this part of the pencil with the back of the pencil. Around its entire surface, there are ball-shaped indents for easier operation of the tool itself, which gives the operator greater freedom of operation, as well as more precise work and safer hold. The middle part of the pencil has two openings, from the front and from the back. The front opening of the middle part is used for setting the microblading blades, while the back opening serves for the positioning and binding of the back of the pencil, i.e. insertion of the needle. The front opening of the middle part may differ in the depth of the opening, depending on which blade is to be used, the golden blade or the black blade.

The back of the pencil is of a more complex shape, which is a variable cylindrical shape on the front that binds to the middle part, while the rear side is of the rectangular shape on which there is a hygienic applicator in the form of three ribs along the longer axis of the pencil with a slight curvature in one direction, which covers about one-eighth of the pencil. On the front part of the back of the pencil there is a conical protrusion with a cut flat part for positioning of the middle part of the pencil, on which the needle is located.

The blade used in the subject invention, which is located on the front part of the middle pencil's portion, is U-shaped and contains 18 tiny needles, the so-called pins. They serve to draw hairs in eyebrows and come in the form of a black or golden blade. Both blades can create hairs that look quite natural, while the main difference is that in the golden blade the pins are extremely sharp, making it easier to penetrate into the skin, even if the skin is thick and problematic; also, it produces extremely thin hairs. Its thinner pins allow for greater flexibility, and a greater difference between the hairs facilitates quicker filling-in with pigment.

The needle used in the subject invention, which is located on the front part of the back of the pencil, consists of three fine pins that are arranged in a circular manner. This needle is used to shade the space between the hairs and is ideal for shading small surfaces. The very material from which the pencil is made is of high quality. At fabrication of the pencil, a special mixture (Addiflex®) with polyphenols is used, which is susceptible to degradation without harmful effects on the environment. When released into the environment, this material degrades spontaneously and assimilates into it without any negative environmental effects. The material itself has been tested and is suitable for use in the fabrication of a pencil is meant to be used in direct contact with human skin.

The design of the pencil is distinguished, in comparison with the existing universal holders, among other things in that that all blades, for microblading and for shading, are situated in one package and in that preliminary sterilization of the universal holder is not necessary, as is neither the opening of additional blades. In this way, operation is accelerated due to the simplicity of switching between one blade and another.

### BRIEF DESCRIPTION THE DRAWINGS

An embodiment of the pencil according to the invention is described in detail below and is shown in the enclosed figures. They show:

FIG. 1 shows the appearance of the pencil with the lid,

FIG. 2 shows the appearance of the disassembled pencil in axonometry,

FIG. 3a shows the longitudinal section of the lid,

FIG. 3b shows the horizontal projection of the pencil lid,

FIG. 4a shows the appearance of the middle part of the pencil, with horizontal projections of the front and back parts,

FIG. 4b shows the cross section of the middle part with the openings for the blade and the needle,

FIG. 5a shows the appearance of the rear part of the pencil,

FIG. 5b shows the cross section of the rear part of the pencil,

FIG. 5c shows the appearance of the hygienic applicator,

FIG. 6a shows the appearance of the blade,

FIG. 6b shows the appearance of the needle.

### DETAILED DESCRIPTION OF THE INVENTION

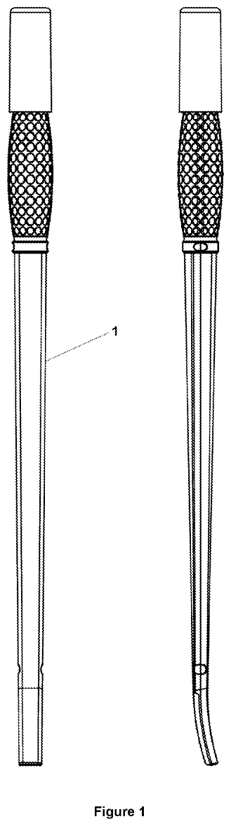

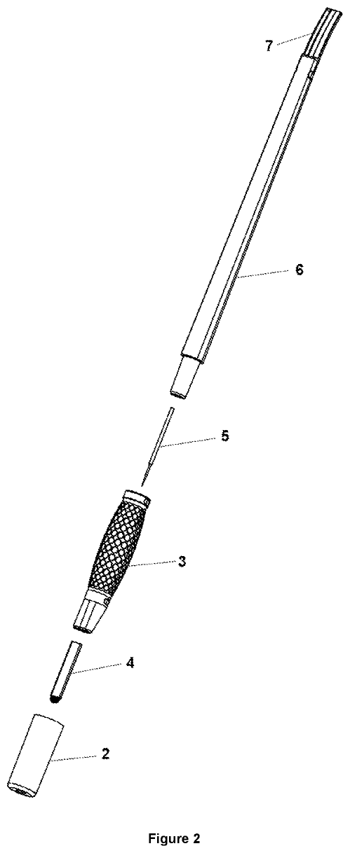

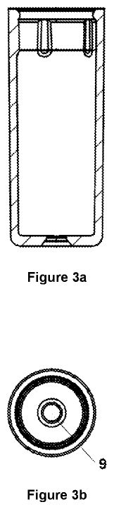

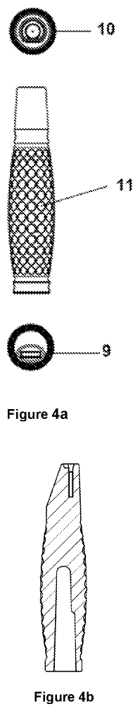

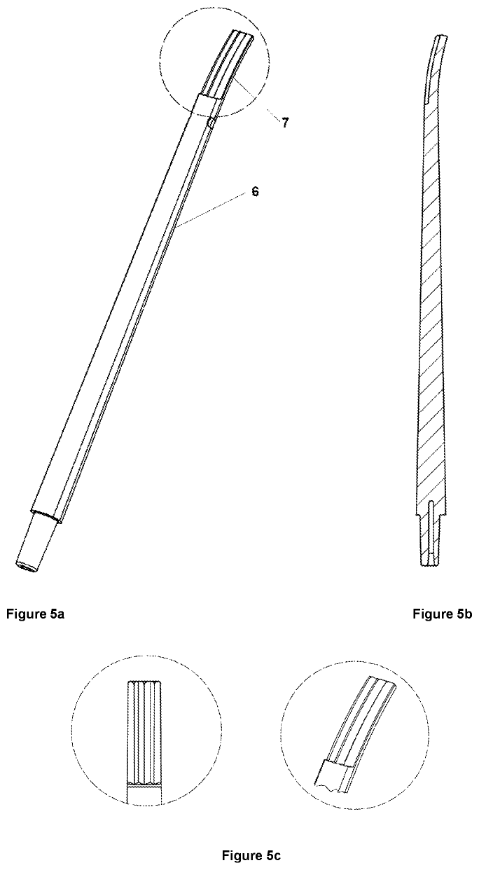

As can be seen from the Figures, and in particular from FIGS. 1 and 2, the pencil (1) comprises a cylindrical lid (2), which is mounted on the middle part (3) of the pencil (1), which on its front part has a blade (4) and on the back there is an opening for inserting a needle (5) located on the front side of the rear part (6), which on its end has a rectangular hygienic applicator (7) which has three ribs. Referring to FIG. 3, viewed from above, the lid (2) itself, which is circular in shape, in its central part has a round opening (8) for penetration of sterile gas, which connects to the front part (9) of the middle part (3) by positioning by means of a ring, which is located along the entire inner rim.

As can be seen from the Figures, the middle part (3) of the pencil (1) is of a barrel-shape with two cylindrical surfaces that give symmetry to the middle part (3). Throughout its entire surface, the middle part 3 has ball-shaped indents (11) for easier grip of pencil (1) during use. The cylindrical front part of the middle part (3) of the pencil is tapered so that the tip of the front part narrows by one-half of the width of the middle part (3) itself, and passes into an elliptical shape. The opening (9) on the front part of the middle part 3, when viewed from above, is elliptical in shape and is used to mount the microblading blades (4), at which the aperture itself for the positioning of the blade is of a rectangular shape. Whereas the opening (10) at the rear of the middle part 3, when viewed from above, is circular in shape, with one part of the circle is cut flat. It serves to position the needle (5) and bonding of the rear part (6). When looking at the cross-section of the middle part (3), the depth of the rear part with the flattened side on one part occupies one-half of the height of the middle part 3, while the depth of the front part corresponds to the length of the blade (4), which can be either 15 mm or 20 mm, depending on which blade is used.

The rear portion (6) of the pencil (1) is on the front of the cylindrical shape where the needle (5) is located and which binds to the middle part (3) of the pencil (1), while on the rear side it narrows and turns into a rectangular shape with a slight curvature in one direction. At the very top of the back part of the pencil (1), there is a hygienic applicator 7, which is also rectangular in shape and on which there are three ribs along the longer axis. The opening (11) of the rear part 6 of the pencil, which has the opening in which the needle (5) is inserted, is of a smaller diameter than the diameter of the middle part 3, so that it can bond, i.e. fit into the middle part (3).

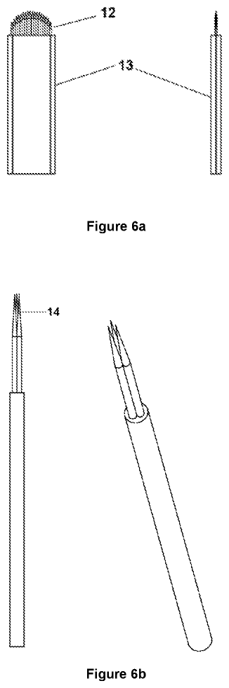

The appearance of the pin (4) which is used for microblading, as can be seen from FIG. 6a, is such that it consists of tiny pins (12), which are of different lengths and which are arranged one next to the other, creating a U-shape. They are made of high quality stainless steel, and the pin holder (13) is made of plastic. The needle (5) that is used for shading, according to FIG. 6b, consists of three needles (14) that are arranged circularly, which in this way form a thicker needle. The material from which the needles are made is also of high quality stainless steel.

1. The multifunctional pencil for eyebrow styling characterized in that having a cylindrical lid (2) which is placed on the middle part (3) of the pencil (1) which has a blade (4) on its front and on its rear there is an opening for the needle insert (5) located on the front side of the rear part (6), which at the end has a rectangular hygienic applicator (7).

2. A multifunctional pencil for eyebrow styling according to claim 1, characterized in that the lid (2) in its central part has a round opening (8) for penetration of the sterile gas.

3. The multifunctional pencil for eyebrow styling according to claim 1, characterized in that the middle part (3) of the pencil (1) is a barrel-shaped, having on its surface ball-shaped indents for easier holding of the Pencil (1).

4. The multifunctional pencil for eyebrow styling according to claim 1, is characterized in that the front part of the middle part (3) is an elliptical shape and is used to position the microblading blade (4).

5. The multifunctional pencil for eyebrow styling according to claim 1, characterized in that the rear part of the middle part is a circular shape and is used to position the needle (6) and connecting the rear part (6).

6. The multifunctional pencil for eyebrow styling according to claim 1, characterized in that the rear part (6) is cylindrical in shape on the front side, while on the rear side it tapers and turns into a rectangular shape with a slight curvature in one direction.

7. The multifunctional pencil for eyebrow styling according to claim 1, characterized in that the front part of the rear part (6) of the pencil (1), containing the shading needle (5), connects to the middle part (3) and such make up one whole.

8. The multifunctional pencil for eyebrow styling according to claim 1, characterized in that the hygienic applicator (7) is of a rectangular shape with three ribs along its longer axis.
